(19) United States
(12) Patent Application Publication
(10) Pub. No.: US 2011/0112145 A1
(43) Pub. Date: May 12, 2011

(54) Salts Of Methyl 2-((R))-(3-Chlorophenyl)((R)-1-((S)-2-(Methylamino)-3((R)-tetrahydro-2H-Pyran-3-YL)Propylcarbamoyl)Piperidin-3-YL)Methoxy)Ethylcarbamate

(76) Inventors: Nicole Marie Deschamps, Research Triangle Park, NC (US); David H. Igo, Research Triangle Park, NC (US); Mark Bryan Mitchell, Research Triangle Park, NC (US)

(21) Appl. No.: 13/001,214

(22) PCT Filed: Jun. 24, 2009
(86) PCT No.: PCT/US09/48389

§ 371 Date: Dec. 23, 2010

Related U.S. Application Data
(60) Provisional application No. 61/075,811, filed on Jun. 26, 2008.

(51) Int. Cl. A61K 31/453; C07D 405/12; A61P 9/12; A61P 9/00; A61P 25/22; A61P 27/06; A61P 9/10

(52) U.S. Cl. 514/326; 546/207

(57) ABSTRACT

Disclosed are salts of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate and pharmaceutical compositions containing the same. Also disclosed are processes for the preparation thereof and methods for use thereof.

### BACKGROUND OF THE INVENTION

In the pursuit of a developable form of a solid, orally-administered pharmaceutical compound, a number of specific features are sought. Although an amorphous form of a pharmaceutical compound may be developed, compounds having high crystallinity are generally preferred. Often such highly crystalline compounds are salts. It is greatly desired that such a salt would also possess the following features: good stability, good aqueous solubility (preferably >1 mg/mL), good in vivo oral bioavailability, and capability of being obtained in good yield (preferably >50%).

International Publication Number WO 2008/036247 describes a series of compounds which are indicated as having inhibitory activity against aspartic proteases, particularly renin, and which are indicated as being useful in the treatment of aspartic protease mediated disorders. Specifically disclosed in that application are the trifluoroacetic acid salt and the pamoate salt (2:1) of methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate.

Although the 2:1 pamoate salt of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate was obtained in good yield and was highly crystalline, this material demonstrated low in vivo oral bioavailability. Following a significant number of screening experiments employing a number of traditional acids, L-(+)-tartaric acid was found to provide a crystalline salt of methyl 2-((R)-(3-chlorophenyl)((R)-1-((S)-2-(methylamino)-3-((R)-tetrahydro-2H-pyran-3-yl)propyl-carbamoyl)piperidin-3-yl)methoxy)ethylcarbamate. However, under a variety of conditions, low yields (15-20%) were obtained of a tartaric acid salt that, although it was crystalline, demonstrated a high degree of amorphous character. Additional experiments with L-(+)-tartaric acid provided a salt form in good yields (−80%), but this material was a complex mixture of at least three different crystalline forms. Accordingly, a developable salt form of methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate was sought.

### SUMMARY OF THE INVENTION

The present invention relates to novel compounds which are salts of methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate. Compounds of the invention are represented by Structure (I):

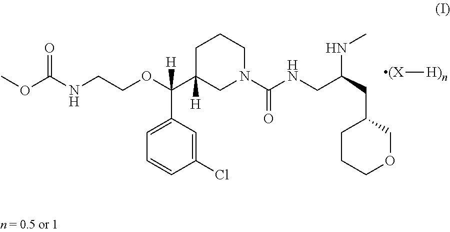

Specifically, compounds where X—H is di-p-toluoyl-L-tartaric acid, N-acetyl-L-phenylalanine, or oxalic acid are described. The compounds of this invention are useful for inhibiting aspartic proteases, particularly renin, and for treating diseases such as hypertension, congestive heart failure, cardiac hypertrophy, cardiac fibrosis, cardiomyopathy post-infarction, nephropathy, vasculopathy and neuropathy, a disease of the coronary vessels, post-surgical hypertension, restenosis following angioplasty, raised intra-ocular pressure, glaucoma, abnormal vascular growth, hyperaldosteronism, an anxiety state, or a cognitive disorder.

### BRIEF DESCRIPTION OF THE DRAWINGS

FIG. 1 shows an X-ray powder diffraction pattern of Compound A-form I.

FIG. 2 shows an X-ray powder diffraction pattern of Compound A-form II.

FIG. 3 shows an X-ray powder diffraction pattern of Compound B-form I.

FIG. 4 shows an X-ray powder diffraction pattern of Compound C-form I.

FIG. 5 shows a differential scanning calorimetry trace of Compound A-form I.

FIG. 6 shows a differential scanning calorimetry trace of Compound A-form II.

FIG. 7 shows a differential scanning calorimetry trace of Compound B-form I.

FIG. 8 shows a differential scanning calorimetry trace of Compound C-form I.

FIG. 9 shows a thermogravimetric analysis trace of Compound A-form I.

FIG. 10 shows a thermogravimetric analysis trace of Compound A-form II.

FIG. 11 shows a thermogravimetric analysis trace of Compound B-form I.

FIG. 12 shows a thermogravimetric analysis trace of Compound C-form I.

### DETAILED DESCRIPTION OF THE INVENTION

Following significant effort involving hundreds of screening experiments, three acid addition salts of methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate were identified that demonstrated the characteristics of a developable solid form. Examples of acids that failed to provide crystalline salts under the conditions employed are acetic acid, (1S)-(+)-10-camphorsulfonic acid, citric acid, ethanesulfonic acid, formic acid, gluconic acid, hippuric acid, hydrobromic acid, L-malic acid, malonic acid, methanesulfonic acid, phosphoric acid, sodium bisulfate, and sulfuric acid. Examples of acids that provided crystalline salts in insufficient quantities to warrant further consideration under the conditions employed are adipic acid, benzoic acid, heptanoic acid, L-(+)-lactic acid, maleic acid, succinic acid, p-toluenesulfonic acid, and p-toluic acid. The acids that provided salts of methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate which demonstrated the characteristics of a developable solid form are di-p-toluoyl-L-tartaric acid, N-acetyl-L-phenylalanine, and oxalic acid. Di-p-toluoyl-L-tartaric acid and N-acetyl-L-phenylalanine have not previously been included in pharmaceutical compounds marketed in the United States. Escitalopram oxalate and oxaliplatin are examples of FDA approved medications that include oxalic acid.

One embodiment of the present invention is directed to a di-p-toluoyl-L-tartaric acid salt of methyl 2-((R)-(3-chlorophenyl)((R)-1-((S)-2-(methylamino)-3-(R)-tetrahydro-2H-pyran-3-yl)-propylcarbamoyl)piperidin-3-yl)methoxy)ethylcarbamate. The invention is further directed to processes for the preparation thereof, pharmaceutical formulations comprising the same, and methods for treating diseases mediated by aspartic proteases by administration of the same, or a pharmaceutical formulation thereof. It is to be understood that the indicated compound may contain a stoichiometric amount of di-p-toluoyl-L-tartaric acid or a variable amount of di-p-toluoyl-L-tartaric acid. When the above indicated compound is named, it is to be understood that solvates (e.g. hydrates) of the compound are also included.

Another embodiment of the present invention is directed to 2:1 methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate di-p-toluoyl-L-tartaric acid (hereinafter “Compound A”), wherein the compound contains a 2:1 ratio of methyl 2-((R)-(3-chlorophenyl)((R)-1-((S)-2-(methyl-amino)-3-((R)-tetrahydro-2H-pyran-3-yl)propylcarbamoyl)piperidin-3-yl)methoxy)ethyl-carbamate to di-p-toluoyl-L-tartaric acid, processes for its preparation, pharmaceutical formulations comprising this compound, and methods for treating diseases mediated by aspartic proteases by administration of this compound, or a pharmaceutical formulation thereof.

Another embodiment of the present invention is directed to an N-acetyl-L-phenylalanine salt of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate. The invention is further directed to processes for the preparation thereof, pharmaceutical formulations comprising the same, and methods for treating diseases mediated by aspartic proteases by administration of the same, or a pharmaceutical formulation thereof. It is to be understood that the indicated compound may contain a stoichiometric amount of N-acetyl-L-phenylalanine or a variable amount of N-acetyl-L-phenylalanine When the above indicated compound is named, it is to be understood that solvates (e.g. hydrates) of the compound are also included.

Another embodiment of the present invention is directed to 1:1 methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate N-acetyl-L-phenylalanine (hereinafter “Compound B”), wherein the compound contains a 1:1 ratio of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate to N-acetyl-L-phenylalanine, processes for its preparation, pharmaceutical formulations comprising this compound, and methods for treating diseases mediated by aspartic proteases by administration of this compound, or a pharmaceutical formulation thereof.

Another embodiment of the present invention is directed to an oxalic acid salt of methyl 2-((R)-(3-chlorophenyl)((R)-1-((S)-2-(methylamino)-3-((R)-tetrahydro-2H-pyran-3-yl)propyl-carbamoyl)piperidin-3-yl)methoxy)ethylcarbamate. The invention is further directed to processes for the preparation thereof, pharmaceutical formulations comprising the same, and methods for treating diseases mediated by aspartic proteases by administration of the same, or a pharmaceutical formulation thereof. It is to be understood that the indicated compound may contain a stoichiometric amount of oxalic acid or a variable amount of oxalic acid. When the above indicated compound is named, it is to be understood that solvates (e.g. hydrates) of the compound are also included.

Another embodiment of the present invention is directed to 1:1 methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate oxalic acid (hereinafter “Compound C”), wherein the compound contains a 1:1 ratio of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate to oxalic acid, processes for its preparation, pharmaceutical formulations comprising this compound, and methods for treating diseases mediated by aspartic proteases by administration of this compound, or a pharmaceutical formulation thereof.

The term “solvates” refers to crystalline forms wherein solvent molecules are incorporated into the crystal lattice during crystallization. Solvates may include water or nonaqueous solvents such as ethanol, dimethyl sulfoxide, acetic acid, ethanolamine, and ethyl acetate. Solvates, wherein water is the solvent molecule incorporated into the crystal lattice, are typically referred to as “hydrates”. Hydrates include stoichiometric hydrates (e.g. a monohydrate), as well as compositions containing variable amounts of water (e.g. a hemi-hydrate).

When a disclosed compound is named, it is to be understood that the compound, including solvates (particularly, hydrates) thereof, may exist in crystalline forms. The compound, or solvates (particularly, hydrates) thereof, may also exhibit polymorphism (i.e. the capacity to occur in different crystalline forms). These different crystalline forms are typically known as “polymorphs.” It is to be understood that when named, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

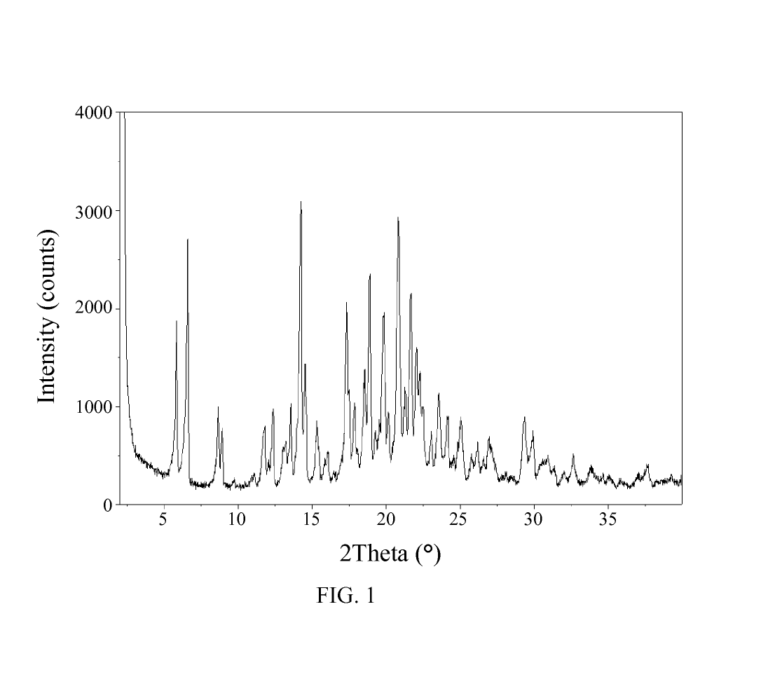

Another embodiment of the present invention is directed to a crystalline form of Compound A (hereinafter “Compound A-form I”), providing an X-ray powder diffraction pattern substantially in accordance with FIG. 1.

Another embodiment of the present invention is directed to a crystalline form of Compound A-form I, providing an X-ray powder diffraction pattern providing diffraction angles (° 2θ) at about 5.9, 6.6, 8.7, 8.9, 11.8, 12.4, 13.1, 13.6, 14.2, 14.5, 15.3, 16.1, 17.3, 17.9, 18.6, 18.9, 19.8, 20.2, 20.8, 21.3, 21.7, 22.1, 22.3, 22.5, 23.0, 23.6, 24.1, 25.1, 25.8, 26.2, 26.9, 29.4, 29.9, and 32.7. More particularly, another embodiment of the present invention is directed to a crystalline form of Compound A-form I, providing an X-ray powder diffraction pattern providing diffraction angles (°2θ) at about 5.9, 6.6, 8.7, 8.9, and 14.2. A further embodiment of the present invention is directed to a crystalline form of Compound A-form I, providing an X-ray powder diffraction pattern providing diffraction angles (°2θ) at about 5.9, 6.6, 14.2, 17.3, 18.9, 19.8, 20.8, and 21.7.

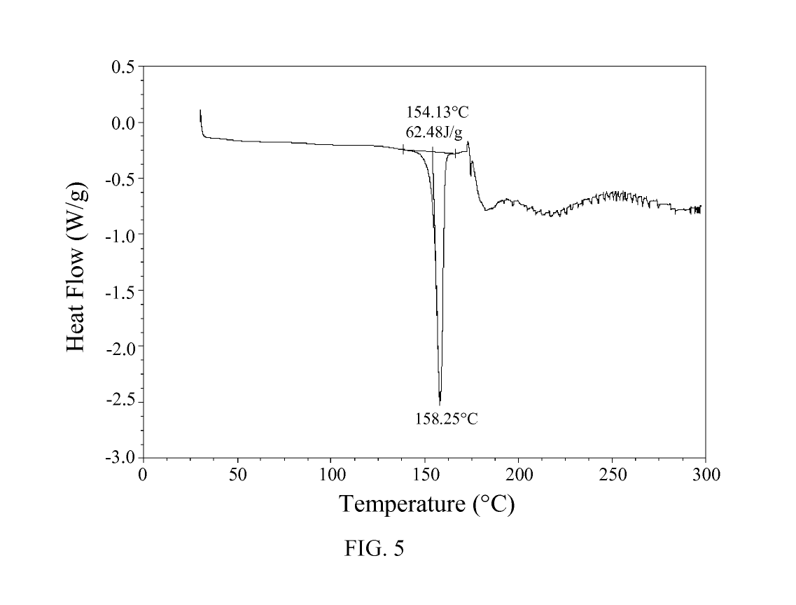

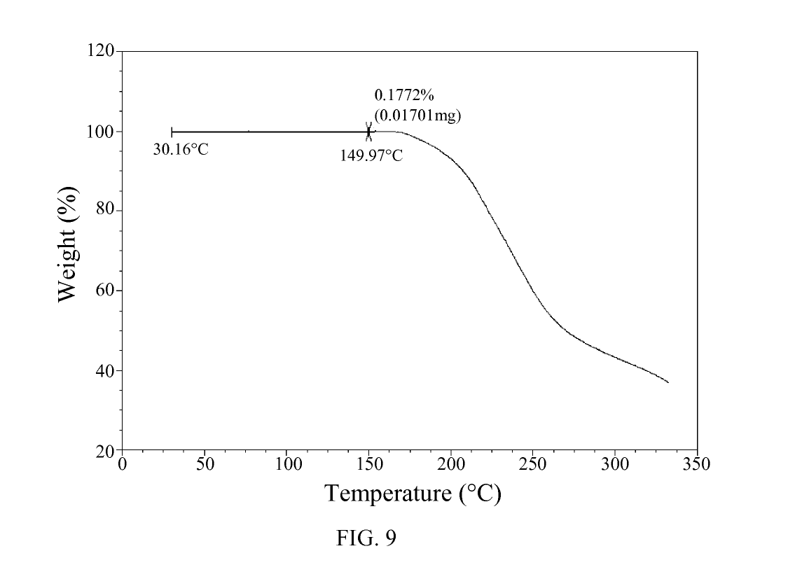

Another embodiment of the present invention is directed to a crystalline form of Compound A-form I, providing a differential scanning calorimetry trace substantially in accordance with FIG. 5 and/or a thermogravimetric analysis trace substantially in accordance with FIG. 9.

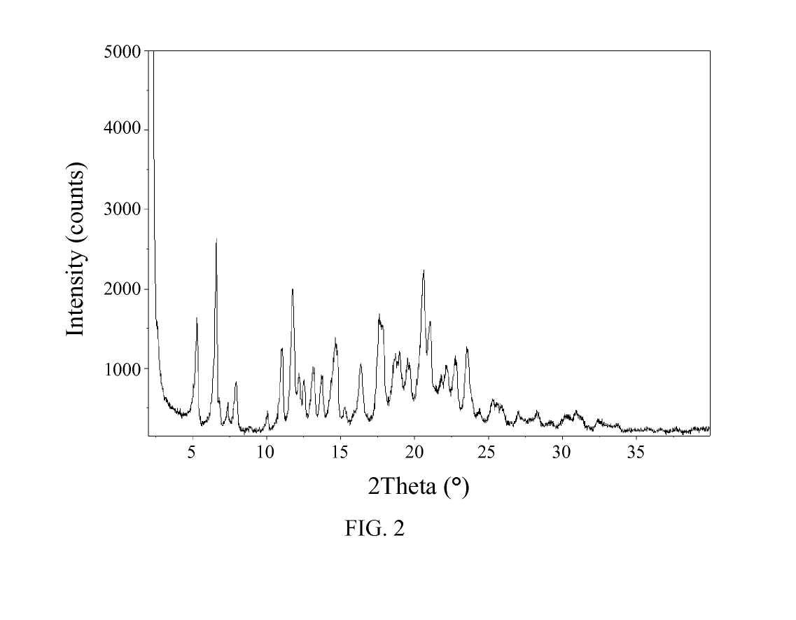

Another embodiment of the present invention is directed to a crystalline form of Compound A (hereinafter “Compound A-form II”), providing an X-ray powder diffraction pattern substantially in accordance with FIG. 2.

Another embodiment of the present invention is directed to a crystalline form of Compound A-form II, providing an X-ray powder diffraction pattern providing diffraction angles (°2θ) at about 5.3, 6.6, 7.4, 7.9, 10.1, 11.1, 11.8, 12.2, 12.5, 13.2, 13.7, 14.7, 15.3, 16.4, 17.6, 17.8, 18.7, 18.9, 19.6, 20.6, 21.1, 22.2, 22.8, and 23.6. More particularly, another embodiment of the present invention is directed to a crystalline form of Compound A-form II, providing an X-ray powder diffraction pattern providing diffraction angles (°2θ) at about 5.3, 6.6, 7.9, 11.1, and 11.8. A further embodiment of the present invention is directed to a crystalline form of Compound A-form II, providing an X-ray powder diffraction pattern providing diffraction angles (°2θ) at about 5.3, 6.6, 11.8, 17.6, 17.8, 20.6, and 21.1.

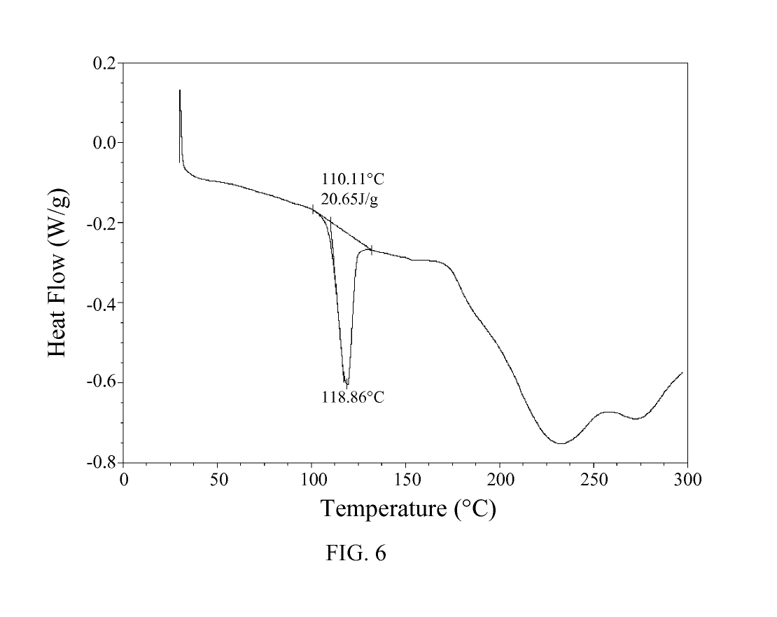

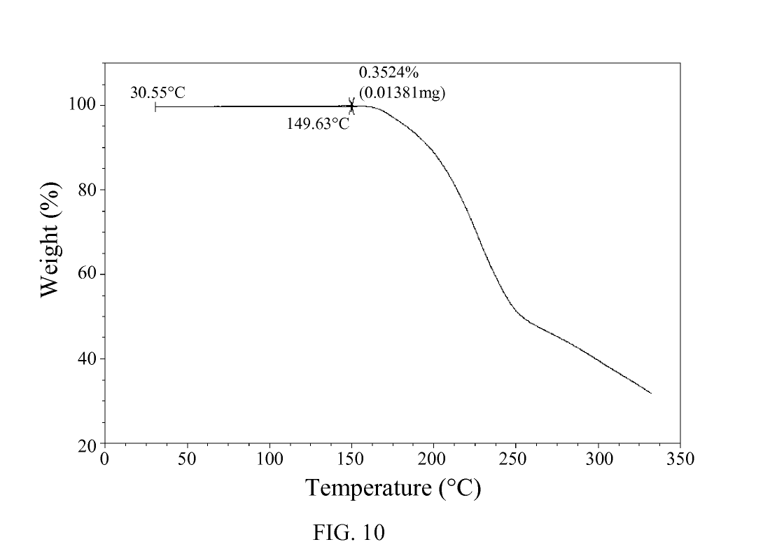

Another embodiment of the present invention is directed to a crystalline form of Compound A-form II, providing a differential scanning calorimetry trace substantially in accordance with FIG. 6 and/or a thermogravimetric analysis trace substantially in accordance with FIG. 10.

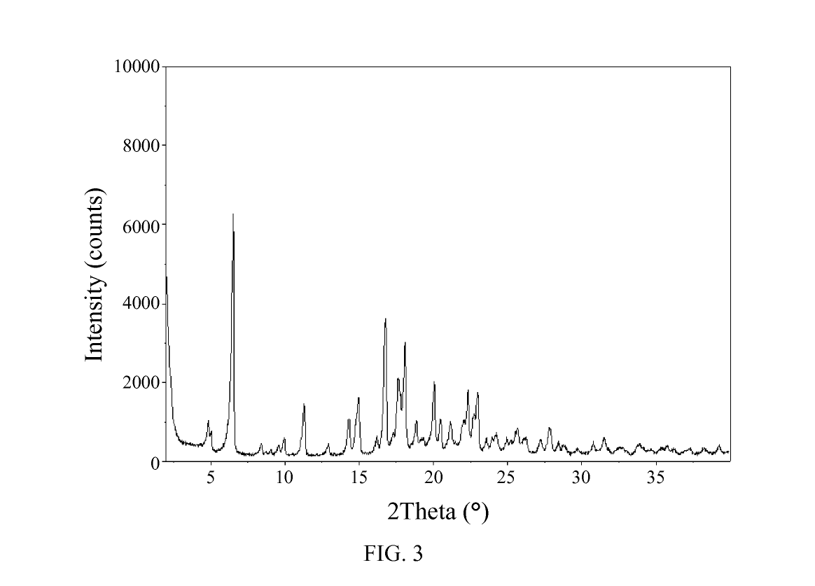

Another embodiment of the present invention is directed to a crystalline form of Compound B (hereinafter “Compound B-form I”), providing an X-ray powder diffraction pattern substantially in accordance with FIG. 3.

Another embodiment of the present invention is directed to a crystalline form of Compound B-form I, providing an X-ray powder diffraction pattern providing diffraction angles (°2θ) at about 4.8, 5.0, 6.5, 8.4, 9.6, 10.0, 11.3, 12.9, 14.3, 15.0, 16.2, 16.8, 17.6, 18.1, 18.9, 20.1, 20.5, 21.2, 22.1, 22.3, 22.7, 23.0, 23.6, 24.2, 24.9, 25.7, 26.2, 27.2, 27.8, and 31.5. More particularly, another embodiment of the present invention is directed to a crystalline form of Compound B-form I, providing an X-ray powder diffraction pattern providing diffraction angles (°2θ) at about 4.8, 6.5, 11.3, 14.3, 15.0, and 16.8. A further embodiment of the present invention is directed to a crystalline form of Compound B-form I, providing an X-ray powder diffraction pattern providing diffraction angles (° 2θ) at about 6.5, 16.8, 18.1, and 20.1.

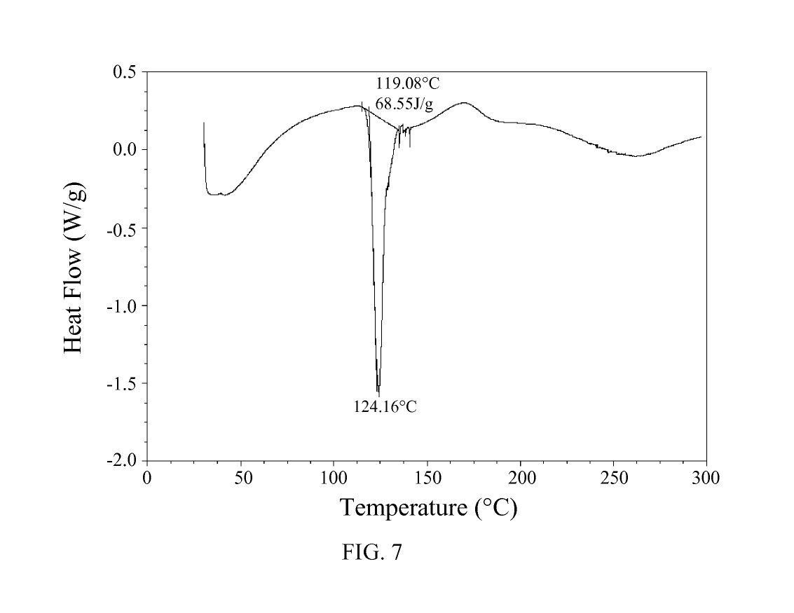

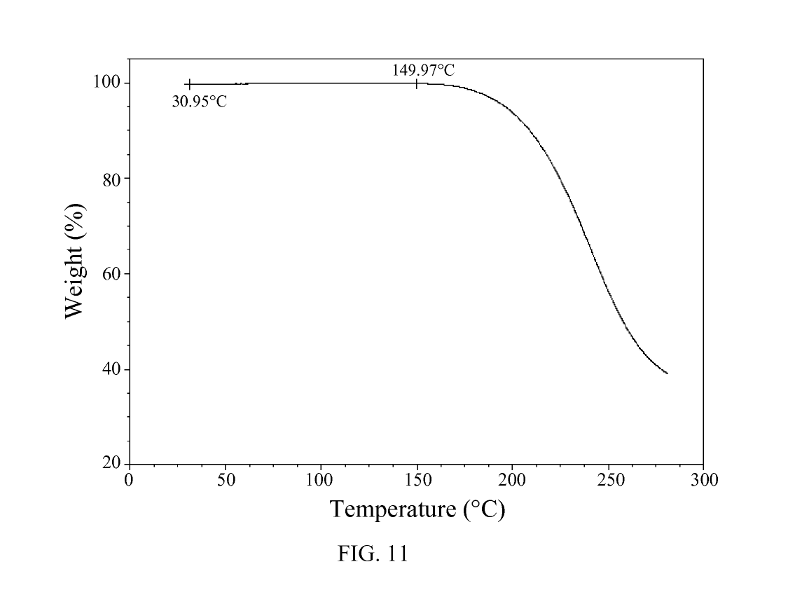

Another embodiment of the present invention is directed to a crystalline form of Compound B-form I, providing a differential scanning calorimetry trace substantially in accordance with FIG. 7 and/or a thermogravimetric analysis trace substantially in accordance with FIG. 11.

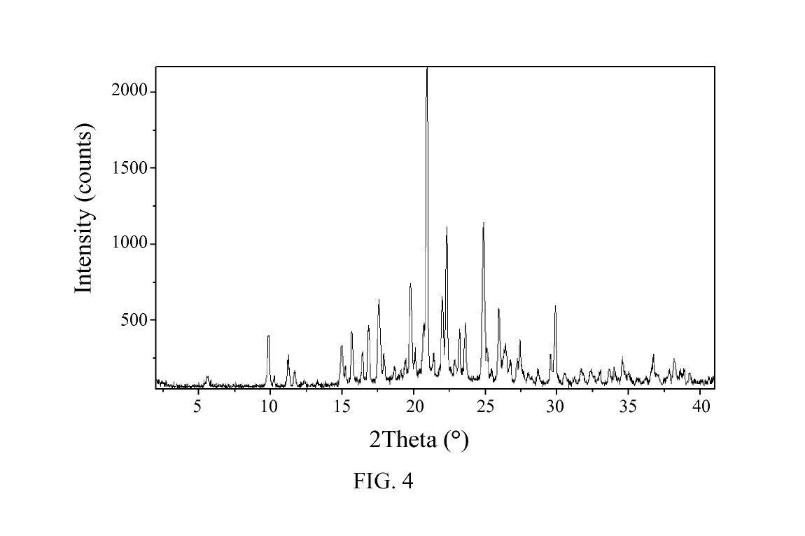

Another embodiment of the present invention is directed to a crystalline form of Compound C (hereinafter “Compound C-form I”), providing an X-ray powder diffraction pattern substantially in accordance with FIG. 4.

Another embodiment of the present invention is directed to a crystalline form of Compound C-form I, providing an X-ray powder diffraction pattern providing diffraction angles (°2θ) at about 9.9, 11.2, 15.0, 15.7, 16.4, 16.8, 17.6, 17.9, 19.8, 20.1, 20.9, 22.0, 22.3, 23.2, 23.6, 24.9, 25.9, 26.3, 27.4, 29.9, and 36.7. More particularly, another embodiment of the present invention is directed to a crystalline form of Compound C-form I, providing an X-ray powder diffraction pattern providing diffraction angles (°2θ) at about 9.9, 15.7, 16.8, 17.6, 17.9, and 19.8. A further embodiment of the present invention is directed to a crystalline form of Compound C-form I, providing an X-ray powder diffraction pattern providing diffraction angles (°2θ) at about 16.8, 17.6, 19.8, 20.9, 22.0, 22.3, and 24.9.

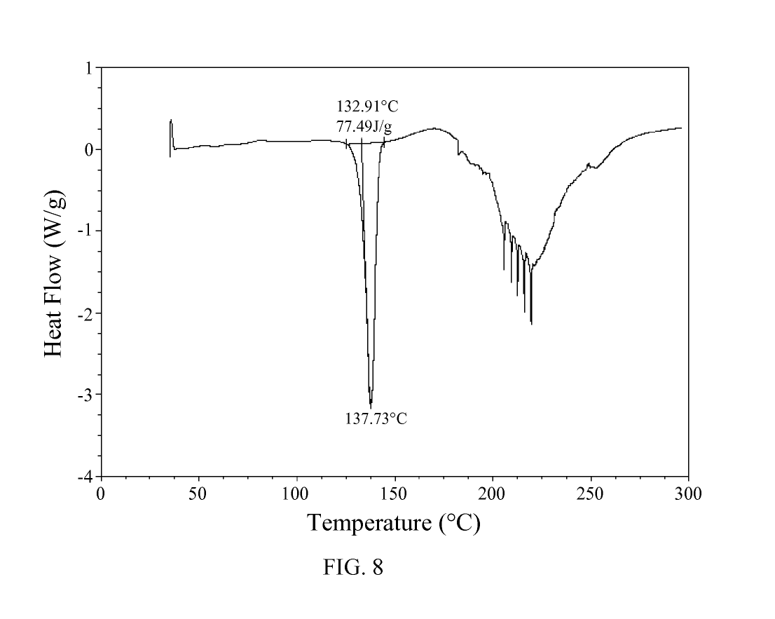

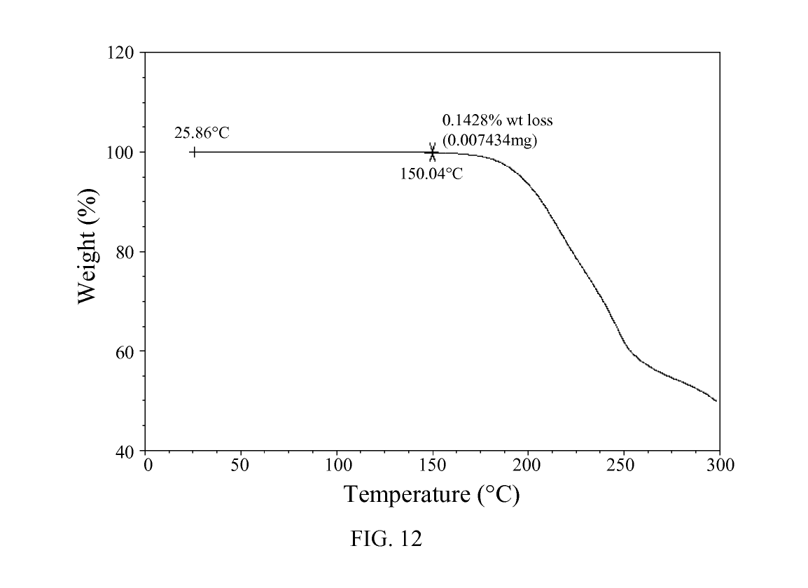

Another embodiment of the present invention is directed to a crystalline form of Compound C-form I, providing a differential scanning calorimetry trace substantially in accordance with FIG. 8 and/or a thermogravimetric analysis trace substantially in accordance with FIG. 12.

It is well known and understood to those skilled in the art that the apparatus employed, humidity, temperature, orientation of the powder crystals, and other parameters involved in obtaining an X-ray powder diffraction (XRPD) pattern may cause some variability in the appearance, intensities, and positions of the lines in the diffraction pattern. An X-ray powder diffraction pattern that is “substantially in accordance” with that of FIG. 1, 2, 3, or 4 provided herein is an XRPD pattern that would be considered by one skilled in the art to represent a compound possessing the same crystal form as the compound that provided the XRPD pattern of FIG. 1, 2, 3, or 4. That is, the XRPD pattern may be identical to that of FIG. 1, 2, 3, or 4, or more likely it may be somewhat different. Such an XRPD pattern may not necessarily show each of the lines of the diffraction patterns presented herein, and/or may show a slight change in appearance, intensity, or a shift in position of said lines resulting from differences in the conditions involved in obtaining the data. A person skilled in the art is capable of determining if a sample of a crystalline compound has the same form as, or a different form from, a form disclosed herein by comparison of their XRPD patterns. For example, one skilled in the art can overlay an XRPD pattern of a sample of a di-p-toluoyl-L-tartaric acid salt of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate, with FIG. 1 and, using expertise and knowledge in the art, readily determine whether the XRPD pattern of the sample is substantially in accordance with the XRPD pattern of Compound A-form I. If the XRPD pattern is substantially in accordance with FIG. 1, the sample form can be readily and accurately identified as having the same form as Compound A-form I. Similarly, a person skilled in the art is capable of determining if a given diffraction angle (expressed in °2θ) obtained from an XRPD pattern is at about the same position as a value presented herein.

“Compound(s) of the invention” means the di-p-toluoyl-L-tartaric acid, N-acetyl-L-phenylalanine, and/or oxalic acid salt of methyl 2-((R)-(3-chlorophenyl)((R)-1-((S)-2-(methylamino)-3-((R)-tetrahydro-2H-pyran-3-yl)propylcarbamoyl)piperidin-3-yl)-methoxy)ethylcarbamate and solvates (particularly, hydrates) thereof, as described herein above, as well as all crystalline forms of said compounds, specifically the crystalline forms defined herein as Compound A-form I, Compound A-form II, Compound B-form I, or Compound C-form I.

Processes for preparing the compounds of the invention are also within the ambit of this invention. To illustrate, a process of the invention comprises mixing a solution of the free base of methyl 2-((R)-(3-chlorophenyl)((R)-1-((S)-2-(methylamino)-3-(R)-tetrahydro-2H-pyran-3-yl)propyl-carbamoyl)piperidin-3-yl)methoxy)ethylcarbamate in an appropriate solvent, such as ethyl acetate or acetone, with an acid selected from di-p-toluoyl-L-tartaric acid, N-acetyl-L-phenylalanine, and oxalic acid, neat or as a solution or suspension in an appropriate solvent, such as ethyl acetate, followed by heating, cooling to room temperature, optionally with further standing or stirring at room temperature, filtering, and drying.

A further process of the invention comprises treating a solution of the free base of methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate in an appropriate solvent, such as ethyl acetate, with a solution of di-p-toluoyl-L-tartaric acid in an appropriate solvent, such as ethyl acetate, followed by heating, eventual cooling to room temperature, standing or stirring at room temperature, filtering, washing with an appropriate solvent, such as ethyl acetate, and drying.

Another process of the invention comprises adding a solution of the free base of methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate in an appropriate solvent, such as ethyl acetate, to a suspension of N-acetyl-L-phenylalanine in an appropriate solvent, such as ethyl acetate, followed by heating, eventual cooling to room temperature, standing or stirring at room temperature, filtering, washing with an appropriate solvent, such as ethyl acetate, and drying.

Another process of the invention comprises treating a solution of the free base of methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate in an appropriate solvent, such as acetone, with oxalic acid, followed by heating, eventual cooling to room temperature, standing or stirring at room temperature, filtering, and drying.

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension, elevated levels of angiotensin I, the product of renin catalyzed cleavage of angiotensinogen, are present. Thus, the compounds of the invention can be used in the treatment of hypertension; heart failure, such as (acute and chronic) congestive heart failure; left ventricular dysfunction; cardiac hypertrophy; cardiac fibrosis; cardiomyopathy (e.g. diabetic cardiac myopathy and post-infarction cardiac myopathy); supraventricular and ventricular arrhythmias; arial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy; diseases of the coronary vessels; proteinuria; albumenuria; post-surgical hypertension; metabolic syndrome; obesity; restenosis following angioplasty; eye diseases and associated abnormalities including raised intra-ocular pressure, glaucoma, retinopathy, abnormal vascular growth and remodeling; angiogenesis-related disorders, such as neovascular age related macular degeneration; hyperaldosteronism; anxiety states; and cognitive disorders (Fisher N. D.; Hollenberg N. K. Expert Opin. Investig. Drugs. 2001, 10, 417-26).

Elevated levels of β-amyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

The invention includes a therapeutic method for treating or ameliorating an aspartic protease mediated disorder in a subject in need thereof comprising administering to a subject in need thereof an effective amount of a compound of the invention.

“Aspartic protease mediated disorder or disease” includes disorders or diseases associated with the elevated expression or overexpression of aspartic proteases and conditions that accompany such diseases.

Administration methods include administering an effective amount of a compound or composition of the invention at different times during the course of therapy or concurrently in a combination form. The methods of the invention include all known therapeutic treatment regimens.

“Effective amount” means that amount of drug substance (i.e. compounds of the present invention) that elicits the desired biological response in a subject. Such response includes alleviation of the symptoms of the disease or disorder being treated. The effective amount of a compound of the invention in such a therapeutic method is from about 0.01 mg/kg/day to about 10 mg/kg/day, preferably from about 0.5 mg/kg/day to 5 mg/kg/day.

An embodiment of the invention includes administering a compound of the invention in a combination therapy (see U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8, the aforementioned article and patents are hereby incorporated by reference) with one or more additional agents for the treatment of hypertension including α-blockers, β-blockers, calcium channel blockers, diuretics, natriuretics, saluretics, centrally acting antihypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

Centrally acting antihypertensives include clonidine, guanabenz, guanfacine and methyldopa.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

An embodiment of the invention includes administering a compound of the invention or a pharmaceutical composition containing the same in a combination therapy with one or more additional agents for the treatment of AIDS reverse transcriptase inhibitors, non-nucleoside reverse transcriptase inhibitors, other HIV protease inhibitors, HIV integrase inhibitors, entry inhibitors (including attachment, co-receptor and fusion inhibitors), antisense drugs, and immune stimulators.

Preferred reverse transcriptase inhibitors are zidovudine, didanosine, zalcitabine, stavudine, lamivudine, abacavir, tenofovir, and emtricitabine.

Preferred non-nucleoside reverse transcriptase inhibitors are nevirapine, delaviridine, and efavirenz.

Preferred HIV protease inhibitors are saquinavir, ritonavir, indinavir, nelfinavir, amprenavir, lopinavir, atazanavir, and fosamprenavir.

Preferred HIV integrase inhibitors are L-870,810 and S-1360.

Entry inhibitors include compounds that bind to the CD4 receptor, the CCR5 receptor or the CXCR4 receptor. Specific examples of entry inhibitors include enfuvirtide (a peptidomimetic of the HR2 domain in gp41) and sifurvitide.

A preferred attachment and fusion inhibitor is enfuvirtide.

An embodiment of the invention includes administering a compound of the invention or a pharmaceutical composition containing the same in a combination therapy with one or more additional agents for the treatment of Alzheimer's disease including tacrine, donepezil, rivastigmine, galantamine, and memantine.

An embodiment of the invention includes administering a compound of the invention or a pharmaceutical composition containing the same in a combination therapy with one or more additional agents for the treatment of malaria including artemisinin, chloroquine, halofantrine, hydroxychloroquine, mefloquine, primaquine, pyrimethamine, quinine, sulfadoxine.

Combination therapy includes co-administration of a compound of the invention and said other agent, sequential administration of the compound of the invention and the other agent, administration of a composition containing the compound of the invention and the other agent, or simultaneous administration of separate compositions containing the compound of the invention and the other agent.

The compounds of the invention may also be administered via a delayed release composition, wherein the composition includes a compound of the invention and a biodegradable slow release carrier (e.g. a polymeric carrier) or a pharmaceutically acceptable non-biodegradable slow release carrier (e.g. an ion exchange carrier).

Biodegradable and non-biodegradable delayed release carriers are well known in the art. Biodegradable carriers are used to form particles or matrices which retain a drug substance(s) (i.e. compounds of the present invention) and which slowly degrade/dissolve in a suitable environment (e.g. aqueous, acidic, basic and the like) to release the drug substance(s). Such particles degrade/dissolve in body fluids to release the drug substance(s) (i.e. compounds of the present invention) therein. The particles are preferably nanoparticles (e.g. in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compounds of the invention may be incorporated for administration orally or by injection in a liquid form, such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds of the invention may be administered parenterally via injection. A parenteral formulation may consist of the drug substance (i.e. compounds of the present invention) dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the drug substance (i.e. compounds of the present invention) in the liquid carrier whereby the final dosage unit contains from 0.005 to 10% by weight of the drug substance (i.e. compounds of the present invention). Other additives include preservatives, isotonizers, solubilizers, stabilizers, and pain-soothing agents. Injectable suspensions may also be prepared, in which case appropriate liquid carriers, suspending agents and the like may be employed.

The compounds of the invention may be administered intranasally using a suitable intranasal vehicle.

The compounds of the invention may also be administered topically using a suitable topical transdermal vehicle or a transdermal patch.

For ocular administration, a pharmaceutical composition containing a compound of the invention is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

The invention includes the use of compounds of the invention for the preparation of a composition for treating or ameliorating an aspartic protease mediated chronic disorder or disease or infection in a subject in need thereof, wherein the composition comprises a mixture of one or more of the compounds of the invention and an optional pharmaceutically acceptable carrier.

The invention further includes the use of compounds of the invention as an active therapeutic substance, in particular in the treatment of aspartic protease mediated disorders. In particular, the invention includes the use of compounds of the invention in the treatment of hypertension, congestive heart failure, cardiac hypertrophy, cardiac fibrosis, cardiomyopathy post-infarction, nephropathy, vasculopathy and neuropathy, a disease of the coronary vessels, post-surgical hypertension, restenosis following angioplasty, raised intra-ocular pressure, glaucoma, abnormal vascular growth, hyperaldosteronism, an anxiety state, or a cognitive disorder.

In another aspect, the invention includes the use of compounds of the invention in the manufacture of a medicament for use in the treatment of the above disorders.

“Pharmaceutically acceptable carrier” means any one or more compounds and/or compositions that are of sufficient purity and quality for use in the formulation of a compound of the invention that, when appropriately administered to a human, do not produce an adverse reaction, and that are used as a vehicle for a drug substance (i.e. compounds of the present invention).

The invention further includes the process for making the composition comprising mixing one or more of the compounds of the invention and an optional pharmaceutically acceptable carrier; and includes those compositions resulting from such a process, which process includes conventional pharmaceutical techniques. For example, a compound of the invention may be nanomilled prior to formulation. A compound of the invention may also be prepared by grinding, micronizing or other particle size reduction methods known in the art. Such methods include, but are not limited to, those described in U.S. Pat. Nos. 4,826,689, 5,145,684, 5,298,262, 5,302,401, 5,336,507, 5,340,564, 5,346,702, 5,352,459, 5,354,560, 5,384,124, 5,429,824, 5,503,723, 5,510,118, 5,518,187, 5,518,738, 5,534,270, 5,536,508, 5,552,160, 5,560,931, 5,560,932, 5,565,188, 5,569,448, 5,571,536, 5,573,783, 5,580,579, 5,585,108, 5,587,143, 5,591,456, 5,622,938, 5,662,883, 5,665,331, 5,718,919, 5,747,001, PCT applications WO 93/25190, WO 96/24336, and WO 98/35666, each of which is incorporated herein by reference. The pharmaceutical compositions of the invention may be prepared using techniques and methods known to those skilled in the art. Some of the methods commonly used in the art are described in Remington's Pharmaceutical Sciences (Mack Publishing Company), the entire teachings of which are incorporated herein by reference.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The dosage form containing the composition of the invention contains an effective amount of the drug substance (i.e. compounds of the present invention) necessary to provide a therapeutic and/or prophylactic effect. The composition may contain from about 5,000 mg to about 0.5 mg (preferably, from about 1,000 mg to about 0.5 mg) of a compound of the invention and may be constituted into any form suitable for the selected mode of administration. The compositions of the invention may be administered in a form suitable for once-weekly or once-monthly administration. Daily administration or post-periodic dosing may also be employed, wherein the composition may be administered about 1 to about 5 times per day.

For oral administration, the composition is preferably in the form of a tablet or capsule containing, e.g. 1000 to 0.5 milligrams of the drug substance (i.e. compounds of the present invention), more specifically 500 mg to 5 mg. Dosages will vary depending on factors associated with the particular patient being treated (e.g. age, weight, diet, and time of administration), the severity of the condition being treated, the compound being employed, the mode of administration, and the strength of the preparation.

The oral composition is preferably formulated as a homogeneous composition, wherein the drug substance (i.e. a compound of the present invention) is dispersed evenly throughout the mixture, which may be readily subdivided into dosage units containing equal amounts of a compound of the invention. Preferably, the compositions are prepared by mixing a compound of the invention with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binding agents include starch, gelatin, natural sugars (e.g. glucose and beta-lactose), corn sweeteners and natural and synthetic gums (e.g. acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Without further elaboration, it is believed that one skilled in the art can, using the preceding description, utilize the present invention to its fullest extent. The following Examples are, therefore, to be construed as merely illustrative and not a limitation of the scope of the present invention in any way.

### Example 1

### Preparation of

### Methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate

A solution of the trifluoroacetic acid salt of methyl {2-[((R)-(3-chlorophenyl){(3R)-1-[({(2S)-2-(methyl-amino)-3-[(3R)-tetrahydro-2H-pyran-3-yl]propyl}amino)carbonyl]-3-piperidinyl}methyl)oxy]ethyl}carbamate (prepared as in WO 2008/036247) (10.0 g, 15.65 mmol) in 200 ml, of dichloromethane was washed successively with 1N aqueous sodium hydroxide, water, and brine. The organic portion was dried over Na₂SO₄ and concentrated in vacuo to afford the title compound as an off-white foam (7.50 g, 91%). ¹H NMR (CD₃OD, 400 MHz) δ ppm 7.38-7.31 (m, 3H), 7.24 (m, 1H), 4.23 (dd, J=13.1, 3.6 Hz, 1H), 4.03 (d, J=8.8 Hz, 1H), 3.84 (m, 3H), 3.64 (s, 3H), 3.42 (ddd, Jₐ=5.8 Hz, J_{b}=7.8 Hz, J_{c}=11.1 Hz, 1H), 3.24-3.30 (m, 5H), 3.16 (dd, Jₐ=6.3 Hz, J_{b}=13.9 Hz, 1H), 3.10 (dd, Jₐ=10 Hz, J_{b}=11 Hz, 1H), 2.88 (m, 2H), 2.66 (m, 1H), 2.42 (s, 3H), 1.97 (m, 1H), 1.75 (m, 2H), 1.65-1.61 (m, 3H), 1.40-1.09 (m, 6H); MS (m/z) 525.3 (M+H⁺).

### Example 2

### Preparation of

### 2:1 methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate di-p-toluoyl-L-tartaric acid (Compound A-form I)

To a solution of di-p-toluoyl-L-tartaric acid (0.048 g, 0.124 mmol) in 0.325 mL of ethyl acetate was added methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (0.130 g, 0.248 mmol) as a solution in 0.325 mL of ethyl acetate. The resulting solution was heated to 50° C. while stirring at 600 RPM. After 80 min., a white solid began to form. After an additional 2 h at 50° C., the sample was cooled to ambient temperature over 6 h and held at ambient temperature overnight. Centrifugation of the sample was followed by removal of the supernatant. The remaining solid was collected and used as seed crystals in a subsequent experiment.

### Example 3

### Preparation of

### 2:1 methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate di-p-toluoyl-L-tartaric acid (Compound A-form I)

To a solution of di-p-toluoyl-L-tartaric acid (0.110 g, 0.284 mmol) in 0.750 mL of ethyl acetate was added methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (0.300 g, 0.572 mmol) as a solution in 0.750 mL of ethyl acetate. The resulting solution was heated to 50° C. with constant stirring (500 RPM). After 90 min., seed crystals (from Example 2) were added to the solution and stirring continued while the mixture was maintained at 50° C. A white precipitate started to appear within approximately 20 min. of adding the seed crystals. The slurry was held at 50° C. for 2 h and subsequently cooled slowly to room temperature. The slurry was left at room temperature overnight. The slurry was filtered, washed with ethyl acetate, and dried in a vacuum oven at 50° C. for approximately 3 h to afford the title compound as a white solid (0.354 g, 86%).

### Example 4

### Preparation of

### 2:1 methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate di-p-toluoyl-L-tartaric acid (Compound A-form I)

To a solution of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (1.0 g, 1.904 mmol) in 2.5 mL of ethyl acetate was added di-p-toluoyl-L-tartaric acid (0.368 g, 0.952 mmol) as a solution in 2.5 mL of ethyl acetate. The resulting solution was stirred at 50° C. for 90 min. At this time, seed crystals (from Example 3) were added. After an additional 10 min. of stirring, a white precipitate formed in such a quantity that stirring was no longer possible. The mixture was then allowed to sit at room temperature for 4 days before it was filtered and washed with ethyl acetate. The solid was dried under vacuum for 2 h at room temperature followed by drying at 50° C. for 3 h to afford the title compound as a white solid (1.24 g, 91%). ¹H NMR (CD₃OD, 400 MHz) δ ppm 8.07 (d, J=8.3 Hz, 2H), 7.36-7.21 (m, 7H), 5.87 (s, 1H), 4.20 (d, J=12.4 Hz, 1H), 4.01 (d, J=8.9 Hz, 1H), 3.87-3.74 (m, 3H), 3.63 (s, 3H), 3.53 (dd, Jₐ=2.4 Hz, J_{b}=14.7 Hz, 1H), 3.41 (ddd, Jₐ=2.5 Hz, J_{b}=4.3 Hz, J_{c}=11 Hz, 1H), 3.29-3.19 (m, 7H), 3.10 (dd, Jₐ=9.5 Hz, J_{b}=11 Hz, 1H), 2.92-2.82 (m, 2H), 2.67 (s, 3H), 2.42 (s, 3H), 1.95 (m, 1H), 1.73 (m, 2H), 1.65-1.57 (m, 3H), 1.43 (t, J=7.0 Hz, 2H), 1.31-1.09 (m, 4H); ¹³C NMR (CD₃OD, 100 MHz) δ ppm 174.1, 167.9, 160.3, 159.6, 145.0, 144.2, 135.6, 131.2, 131.0, 129.2, 129.1, 128.4, 127.1, 84.9, 76.9. 74.0, 69.4, 69.1, 59.0, 52.5, 48.1, 45.8, 43.9, 41.9, 41.5, 33.6, 31.7, 31.2, 29.7, 28.0, 26.4, 25.5, 21.7; MS (m/z) 525.0 (M+H⁺). The X-ray powder diffraction pattern of this material is shown in FIG. 1 and a summary of the diffraction angles, d-spacings, and relative intensities is given in Table I. Data were acquired according to the following parameters:
Scan range: 2-40° 2θ
Generator power: 40 kV, 40 mA
Radiation Source: Cu Ka
Scan type: Continuous
Time per step: 10 s
Step size: 0.017°2θ per step
Sample Rotation: is revolution time
Incident Beam optics: 0.04 radian soller slits, 0.25° divergent slit, 10 mm beam mask, 0.5° anti-scatter slit
Diffracted Beam optics: fixed slits (X'celerator module), 0.04 radian soller slits
Detector Type Philips X'Celerator RTMS (Real Time Multi Strip)

TABLE I

| Diff. Angle [°2θ] | d-spacing [Å] | Rel. Intensity [%] |
|---|---|---|
| 5.9 | 15.11 | 54.8 |
| 6.6 | 13.41 | 85.4 |
| 8.7 | 10.21 | 27.9 |
| 8.9 | 9.91 | 20.2 |
| 11.8 | 7.51 | 20.7 |
| 12.4 | 7.17 | 26.5 |
| 13.1 | 6.74 | 13.4 |
| 13.6 | 6.53 | 28.8 |
| 14.2 | 6.22 | 100.0 |
| 14.5 | 6.09 | 42.8 |
| 15.3 | 5.78 | 21.6 |
| 16.1 | 5.52 | 11.7 |
| 17.3 | 5.12 | 64.1 |
| 17.9 | 4.97 | 28.2 |
| 18.6 | 4.78 | 40.5 |
| 18.9 | 4.70 | 73.8 |
| 19.8 | 4.48 | 58.8 |
| 20.2 | 4.41 | 25.4 |
| 20.8 | 4.26 | 93.8 |
| 21.3 | 4.17 | 32.6 |
| 21.7 | 4.10 | 66.4 |
| 22.1 | 4.03 | 48.0 |
| 22.3 | 3.99 | 38.7 |
| 22.5 | 3.95 | 26.2 |
| 23.0 | 3.86 | 18.0 |
| 23.6 | 3.77 | 31.0 |
| 24.1 | 3.69 | 23.0 |
| 25.1 | 3.55 | 22.7 |
| 25.8 | 3.45 | 8.8 |
| 26.2 | 3.41 | 13.6 |
| 26.9 | 3.31 | 16.0 |
| 29.4 | 3.04 | 22.9 |
| 29.9 | 2.99 | 16.1 |
| 32.7 | 2.74 | 9.0 |

The differential scanning calorimetry trace of this material is shown in FIG. 5. Data were acquired on a TA instruments Q1000 Differential Scanning calorimeter. The sample was heated from 30° C. to 300° C. at 10° C./min. The thermogravimetric analysis trace of this material is shown in FIG. 9. Data were acquired on a TA instruments Q500 Thermogravimetric Analyzer. The sample was heated from 30° C. to 300° C. at 10° C./min.

### Example 5

### Preparation of

### 2:1 methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate di-p-toluoyl-L-tartaric acid (Compound A-form II)

To a solution of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (14.0 g, 26.7 mmol) in 35 mL of ethyl acetate was added di-p-toluoyl-L-tartaric acid (5.15 g, 13.33 mmol) as a solution in 35 mL of ethyl acetate. The resulting solution was stirred at 50° C. for 54 min. The mixture was then allowed to stand at room temperature overnight before it was filtered and washed with ethyl acetate. The solid was lyophilized over the weekend followed by drying in vacuo at 50° C. for 3 hours to afford the title compound as a white solid (17.56 g, 92%). The X-ray powder diffraction pattern of this material is shown in FIG. 2 and a summary of the diffraction angles, d-spacings, and relative intensities is given in Table II.

TABLE II

| Diff. Angle [°2θ] | d-spacing [Å] | Rel. Intensity [%] |
|---|---|---|
| 5.3 | 16.71 | 52.8 |
| 6.6 | 13.41 | 100.0 |
| 7.4 | 12.00 | 12.1 |
| 7.9 | 11.15 | 25.1 |
| 10.1 | 8.80 | 9.2 |
| 11.1 | 8.01 | 45.4 |
| 11.8 | 7.53 | 78.4 |
| 12.2 | 7.26 | 31.1 |
| 12.5 | 7.07 | 26.9 |
| 13.2 | 6.73 | 33.0 |
| 13.7 | 6.45 | 28.7 |
| 14.7 | 6.04 | 49.4 |
| 15.3 | 5.80 | 12.8 |
| 16.4 | 5.41 | 37.4 |
| 17.6 | 5.03 | 62.5 |
| 17.8 | 4.98 | 58.2 |
| 18.7 | 4.75 | 41.2 |
| 18.9 | 4.69 | 39.5 |
| 19.6 | 4.53 | 37.8 |
| 20.6 | 4.31 | 87.5 |
| 21.1 | 4.22 | 59.2 |
| 22.2 | 4.01 | 35.2 |
| 22.8 | 3.90 | 38.2 |
| 23.6 | 3.78 | 44.7 |

The differential scanning calorimetry trace of this material is shown in FIG. 6. The thermogravimetric analysis trace of this material is shown in FIG. 10. Data were acquired as in Example 4.

### Example 6

### Preparation of

### 1:1 methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate N-acetyl-L-phenylalanine (Compound B-form I)

To a suspension of N-acetyl-L-phenylalanine (0.051 g, 0.248 mmol) in 0.325 mL of ethyl acetate was added methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (0.130 g, 0.248 mmol) as a solution in 0.325 mL of ethyl acetate. The resulting clear solution was heated to 50° C. while stirring at 600 RPM. After 80 min, heptane (0.1 mL) was added to the solution and a white solid began to form within approximately 20 min. After 2 h at 50° C., the sample was cooled to ambient temperature over a period of 6 h and held at ambient temperature overnight. Centrifugation of the sample was followed by removal of the supernatant. The remaining solid was collected and used as seed crystals in a subsequent experiment.

### Example 7

### Preparation of

### 1:1 methyl 2-((R)-(3-chlorophenyl)((R)-1-(S)-2-(methylamino)-3-((R)-tetrahydro-2H-pyran-3-yl)propylcarbamoyl)piperidin-3-yl)methoxy)ethylcarbamate N-acetyl-L-phenylalanine (Compound B-form I)

To a suspension of N-acetyl-L-phenylalanine (0.207 g, 1.00 mmol) in 0.750 mL of ethyl acetate was added methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (0.300 g, 0.571 mmol) as a solution in 0.750 mL of ethyl acetate. The vial was heated to 50° C. with constant stirring (500 RPM) resulting in a clear solution. After 90 min, seed crystals (from Example 6) were added to the solution and stirring continued while the mixture was maintained at 50° C. A heavy white precipitate started to form within approximately 20 min. of adding the seed crystals. An additional aliquot of ethyl acetate (0.750 mL) was added to facilitate stirring, and the slurry was held at 50° C. for 2 h. The slurry was then cooled slowly to room temperature and subsequently held overnight. The slurry was filtered, washed with ethyl acetate, and air dried on a vacuum filter for 30 min. to afford the title compound as a white solid (0.288 g, 69%). The X-ray powder diffraction pattern of this material is shown in FIG. 3 and a summary of the diffraction angles, d-spacings, and relative intensities is given in Table III.

TABLE III

| Diff. Angle [°2θ] | d-spacing [Å] | Rel. Intensity [%] |
|---|---|---|
| 4.8 | 18.29 | 11.2 |
| 5.0 | 17.58 | 7.0 |
| 6.5 | 13.58 | 100.0 |
| 8.4 | 10.53 | 4.3 |
| 9.6 | 9.25 | 3.9 |
| 10.0 | 8.88 | 7.2 |
| 11.3 | 7.84 | 21.6 |
| 12.9 | 6.85 | 4.4 |
| 14.3 | 6.19 | 15.3 |
| 15.0 | 5.92 | 24.6 |
| 16.2 | 5.48 | 7.8 |
| 16.8 | 5.29 | 58.4 |
| 17.6 | 5.03 | 32.3 |
| 18.1 | 4.91 | 47.5 |
| 18.9 | 4.71 | 14.5 |
| 20.1 | 4.43 | 31.3 |
| 20.5 | 4.34 | 15.1 |
| 21.2 | 4.20 | 14.0 |
| 22.1 | 4.03 | 14.6 |
| 22.3 | 3.98 | 26.5 |
| 22.7 | 3.91 | 17.1 |
| 23.0 | 3.87 | 26.5 |
| 23.6 | 3.77 | 6.8 |
| 24.2 | 3.67 | 9.0 |
| 24.9 | 3.57 | 7.1 |
| 25.7 | 3.47 | 11.1 |
| 26.2 | 3.40 | 7.3 |
| 27.2 | 3.28 | 6.1 |
| 27.8 | 3.21 | 11.4 |
| 31.5 | 2.84 | 7.3 |

The differential scanning calorimetry trace of this material is shown in FIG. 7. The thermogravimetric analysis trace of this material is shown in FIG. 11. Data were acquired as in Example 4.

### Example 8

### Preparation of

### 1:1 methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate oxalic acid (Compound C-Form I)

To a solution of oxalic acid (2 equivalents) in acetone was added methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (1 equivalent). The mixture was sonicated for approximately 1 min., which resulted in a clear solution. Hexanes were added to the solution until a haze formed. The mixture was then back-titrated with acetone until a clear solution persisted. The solution was left at ambient temperature for 10 d, during which time a precipitate had formed. Centrifugation of the sample was followed by removal of the supernatant. The remaining solid was collected and used as seed crystals in a subsequent experiment.

### Example 9

### Preparation of

### 1:1 methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate oxalic acid (Compound C-Form I)

To a solution of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (2.43 g, 4.62 mmol) in 50.0 mL of acetone was added oxalic acid (0.457 g, 5.08 mmol). The resulting solution was heated to 40° C. and seed crystals (from Example 8) were added. A slurry resulted and stirring continued at 40° C. After approximately 1 h the slurry became noticeably thicker and stirring was continued for approximately an additional 3 h. The slurry was cooled to 20° C. at 0.25° C./min and allowed to stir at 20° C. for three days. The solids were filtered and dried at 50° C. under vacuum over the weekend to afford the title compound as a white solid (1.49 g, 52%). The X-ray powder diffraction pattern of this material is shown in FIG. 4 and a summary of the diffraction angles, d-spacings, and relative intensities is given in Table IV.

TABLE IV

| Diff. Angle [°2θ] | d-spacing [Å] | Rel. Intensity [%] |
|---|---|---|
| 9.9 | 8.97 | 17.6 |
| 11.2 | 7.89 | 8.4 |
| 15.0 | 5.92 | 12.9 |
| 15.7 | 5.66 | 18.9 |
| 16.4 | 5.40 | 11.3 |
| 16.8 | 5.27 | 31.6 |
| 17.6 | 5.05 | 36.0 |
| 17.9 | 4.96 | 15.0 |
| 19.8 | 4.49 | 31.4 |
| 20.1 | 4.43 | 14.3 |
| 20.9 | 4.25 | 100.0 |
| 22.0 | 4.04 | 45.0 |
| 22.3 | 3.99 | 53.3 |
| 23.2 | 3.84 | 20.5 |
| 23.6 | 3.78 | 24.8 |
| 24.9 | 3.58 | 51.7 |
| 25.9 | 3.44 | 24.2 |
| 26.3 | 3.39 | 18.8 |
| 27.4 | 3.26 | 16.9 |
| 29.9 | 2.99 | 21.2 |
| 36.7 | 2.45 | 9.3 |

The differential scanning calorimetry trace of this material is shown in FIG. 8. The thermogravimetric analysis trace of this material is shown in FIG. 12. Data were acquired as in Example 4.

### Example 10

### Preparation of

### methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate fumaric acid (hereinafter—“Compound D”)

To a solution of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (0.0342 g, 0.065 mmol) in ethanol (1.0 mL) was added fumaric acid (0.0076 g, 0.065 mmol). The resulting solution was stirred until totally clear and the solvent was removed in vacuo. The residue was dissolved in water (0.5 mL) and lyophilized to give the title compound as a white solid (0.03024 g, 72%).

### Example 11

### Oral Bioavailability

The 2:1 pamoate salt of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate (hereinafter—“Compound E”) can be prepared as described in International Publication Number WO 2008/036247.

### Oral Bioavailability in Sprague-Dawley Rats

Oral pharmacokinetic data in male Sprague-Dawley rats was obtained for a solution formulation of Compound D and a suspension formulation of micronized Compound E. Compound D was administered by oral gavage as a clear, colorless solution in a formulation with 0.5% methylcellulose in water at a dose of 10 mg/kg (5 mL of dose solution per kg). Blood was sampled at the following time intervals: 0, 5, 15, 30, 60, 120, 180, 240, 360, 480, and 1440 min. Compound E was administered as a suspension by gastric bolus in a formulation with 1% methylcellulose in water at a dose of 5 mg/kg (10 mL of dose solution per kg). Blood (50 μL) was sampled at the following time intervals: 0, 20, 40, 60, 120, 180, 240, 360, 480, 720, 960, 1200, and 1440 min.

### Oral Bioavailability in Beagle Dogs

Oral pharmacokinetic data in male Beagle dogs was obtained for a solution formulation of Compound D and capsule formulations of Compound A-form I, Compound B-form I, and Compound C-form I. Compound D was administered by oral gavage as a clear, colorless solution in water containing 2% DMSO (final pH=3.5), which was filtered, using a 0.22 μm MILLEX-GV filter, prior to administration. The above noted Compounds A, B, and C were each administered at a dose of 5 mg/kg in gelatin capsules (1.37 mL). Blood (50 μL) was sampled at the following time intervals: 0, 20, 40, 60, 120, 180, 240, 360, 480, 600, and 1440 min.

The concentration of each compound was quantified by LC/MS/MS analysis of an aliquot (25 μL blood+25 μL water) of these samples and the overall blood exposure reported as the Dose-Normalized Area Under the Curve (DNAUC) from a concentration versus time plot and expressed in the units kilogram hours per liter (kg·h/L). All oral bioavailabilities were calculated by dividing the DNAUC from an oral segment by the DNAUC from an i.v. segment and multiplying by 100. The data are summarized in Table V below.

TABLE V

| | | | DNAUC | Oral |
|---|---|---|---|---|
| Species | Compound | Dose Form | (kg · h/L) | Bioavailability (%) |
| Rat | D | Solution | 0.048 | 37 |
| | E | Suspension | 0.0067 | 5 |
| Dog | A | Capsule | 0.38 | 35 |
| | B | Capsule | 0.40 | 37 |
| | C | Capsule | 0.64 | 59 |
| | D | Solution | 0.46 | 43 |

1. A compound which is a di-p-toluoyl-L-tartaric acid salt of methyl 2-((R)-(3-chlorophenyl)((R)-1-((S)-2-(methylamino)-3-(R)-tetrahydro-2H-pyran-3-yl)propylcarbamoyl)piperidin-3-yl)methoxy)ethylcarbamate.

2. The compound according to claim 1, wherein said salt is a 2:1 ratio of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate to di-p-toluoyl-L-tartaric acid.

3. The compound according to claim 2, wherein said compound is a crystalline compound that provides an X-ray powder diffraction pattern substantially in accordance with FIG. 1.

4. The compound according to claim 2, wherein said compound is a crystalline compound that provides an X-ray powder diffraction pattern substantially in accordance with FIG. 2.

5. A compound which is an N-acetyl-L-phenylalanine salt of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate.

6. The compound according to claim 5, wherein said salt is a 1:1 ratio of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate to N-acetyl-L-phenylalanine.

7. The compound according to claim 6, wherein said compound is a crystalline compound that provides an X-ray powder diffraction pattern substantially in accordance with FIG. 3.

8. A compound which is an oxalic acid salt of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate.

9. The compound according to claim 8, wherein said salt is a 1:1 ratio of methyl 2-((R)-(3-chlorophenyl)((R)-1-((S)-2-(methylamino)-3-((R)-tetrahydro-2H-pyran-3-yl)propylcarbamoyl)piperidin-3-yl)methoxy)ethylcarbamate to oxalic acid.

10. The compound according to claim 9, wherein said compound is a crystalline compound that provides an X-ray powder diffraction pattern substantially in accordance with FIG. 4.

11. A pharmaceutical composition comprising the compound according to claim 1, claim 5 or claim 8 and a pharmaceutically acceptable carrier.

12. The pharmaceutical composition of claim 11, further comprising an α-blocker, β-blocker, calcium channel blocker, diuretic, natriuretic, saluretic, centrally acting antihypertensive, angiotensin converting enzyme inhibitor, dual angiotensin converting enzyme and neutral endopeptidase inhibitor, an angiotensin-receptor blocker, dual angiotensin-receptor blocker and endothelin receptor antagonist, aldosterone synthase inhibitor, aldosterone-receptor antagonist, or endothelin receptor antagonist.

13. A method of antagonizing one or more aspartic proteases in a subject in need thereof, comprising administering to the subject an effective amount of the compound according to claim 1, claim 5 or claim 8.

14. The method of claim 13, wherein the aspartic protease is renin.

15. A method for treating an aspartic protease mediated disorder in a subject comprising administering to the subject an effective amount of the compound according to claim 1, claim 5 or claim 8.

16. The method of claim 15, wherein said disorder is hypertension, congestive heart failure, cardiac hypertrophy, cardiac fibrosis, cardiomyopathy post-infarction, nephropathy, vasculopathy and neuropathy, a disease of the coronary vessels, post-surgical hypertension, restenosis following angioplasty, raised intra-ocular pressure, glaucoma, abnormal vascular growth, hyperaldosteronism, an anxiety state, or a cognitive disorder.Z

17. The method of claim 15, further comprising administering one or more additional agents selected from the group consisting of an α-blockers, a β-blocker, a calcium channel blocker, a diuretic, an angiotensin converting enzyme inhibitor, a dual angiotensin converting enzyme and neutral endopeptidase inhibitor, an angiotensin-receptor blocker, dual angiotensin-receptor blocker and endothelin receptor antagonist, a aldosterone
synthase inhibitor, a aldosterone-receptor antagonist, and an endothelin receptor antagonist.

18. The method of claim 15, wherein the aspartic protease is β-secretase.

19. The method of claim 15, wherein the aspartic protease is plasmepsin.

20. The method of claim 15, wherein the aspartic protease is HIV protease.
